(19) United States
(12) Patent Application Publication
(10) Pub. No.: US 2010/0008874 A1
(43) Pub. Date: Jan. 14, 2010

(54) SUBSTITUTED PYRIMIDINES, PROCESS FOR THEIR PRODUCTION AND THEIR USE AS EFFECTIVE ABSORBENTS OF UV IRRADIATION

(75) Inventors: Enk David Claes, Jerusalem (IL); Morris Srebnik, Mevasseret Zion (IL)

Correspondence Address: BROOKS KUSHMAN P.C., 1000 TOWN CENTER, TWENTY-SECOND FLOOR, SOUTHFIELD, MI, 48075, US

(73) Assignee: YISSUM RESEARCH DEVELOPMENT COMPANY, Jerusalem (IL)

(21) Appl. No.: 12/297,106

(22) PCT Filed: Apr. 11, 2007
(86) PCT No.: PCT/IL07/00471

§ 371 Date: May 13, 2009

Related U.S. Application Data
(60) Provisional application No. 60/790,807, filed on Apr. 11, 2006.

(51) Int. Cl. A61K 8/49; C07D 239/46

(52) U.S. Cl. 424/60; 544/311; 544/319; 424/59

(57) ABSTRACT

The present invention discloses substituted pyrimidines, processes for their synthesis and their use as effective sun-protecting agents either alone or in combination with other known sun-protecting agents.

### FIELD OF THE INVENTION

The invention relates to novel substituted pyrimidines, processes for their synthesis and their use as ultraviolet-absorbing agents.

### BACKGROUND OF THE INVENTION

Exposure to ultraviolet radiation (UVR) from the sun plays a causal role hi acute and chronic skin damage such as sunburn, skin cancer, immunosuppression, and photoaging of the skin. These consequences of sun exposure are attracting considerable attention due to an alarming increase in the incidence of sun-related skin cancers. Major culprits of increased sun-related morbidity include changes in life style with more time spent in outdoor recreational activities resulting in significant augmentation in the amount of UVR received and depletion of stratospheric ozone, which is the Earth's protection layer against hazardous radiation. To amend for these dangerous developments, a sun avoidance strategy has been advocated in which the topical application of sunscreens constitutes a cornerstone. However, the increased use of sunscreens raises several concerns: Most sunscreens do not effectively filter out all the detrimental wavelengths of sun light. Second, even though sunscreens prevent sunburn, little is known regarding the threshold or dose-response for UVR-induced effects on other endpoints such as immune suppression and DNA damage. Finally, there is increasing body of evidence that presently used topical sunscreens might undergo UV-induced photooxidation and form potentially toxic metabolites.

Search for a new generation of sunscreens stems from the various drawbacks the present sunscreen agents possess, including photo- and nonphotoinduced skin sensitivity and photogenotoxicity. Naturally occurring UV filters in the form of pigments are abundant and might constitute attractive candidates for new effective and nontoxic sunscreens. In addition to melanin and flavonoides, they include scytonemins found in cyanobacteria with a recently elucidated structure (Proteau et al (1993) Experimentia 49:825-829). This pigment, the first shown to be an effective photostable UV shield in prokaryotes, is a dimeric molecule of indole and phenol subunits. The scytonemin absorbs strongly and broadly in the spectral region of 325-425 nm (UVA) but also has an absorption in the UVB (280-320 nm) and LWC (<250 nm) regions (U.S. Pat. No. 5,461,070). Mycosporine is another family of water-soluble, ultra violet-absorbing metabolites found in cyanobacteria with an UV absorption peak in the UVB range. The elucidated structure of mycosporine is cyclohexenone chromophore conjugated with the nitrogen of an amino acid or an amino alcohol. A variety of specific mycosporin amino acids were identified and their distribution in various groups has been described (Karentz et al. (1991) Marine Biology 108, 157-166).

### SUMMARY OF THE INVENTION

The present invention is based on the findings of a novel family of substituted pyrimidines that can absorb ultra violet radiation.

Thus the present invention is directed to a compound of formula (I):

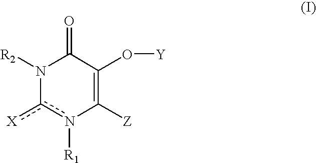

wherein [symbol] denotes an optional double bond, where one of the two optional double bonds being a double bond and the other a single bond; R₁ is null, hydrogen or C₁₋₆alkyl which may be optionally substituted with halogen; R₂ is hydrogen or C₁₋₆alkyl which may be optionally substituted with halogen; X is hydrogen, C₁₋₄alkyl, S—CH₃, SH or ═S; Y is hydrogen, optionally substituted tetrahydropyran, tetrahydrothiopyran, dithiane, or an optionally substituted aryl or heteroaryl; and Z is (a) NH₂; (b) hydrogen (c) —N═NAr, Ar being optionally substituted aryl group.

C₁₋₆allyl is branched or straight chain alkyl groups and may be methyl, ethyl, propyl, isopropyl, butyl, secbutyl, tertbutyl, pentyl, neopentyl, or hexyl. C₁₋₄allyl may be methyl, ethyl, propyl, isopropyl, butyl, secbutyl or tertbutyl that may be partially halogenated, the halogen selected from fluorine, chlorine, bromine, iodine.

Substituents are halogens, straight or branched C₁₋₆alkyl groups optionally partially halogentated. Halogens are selected from fluorine, chlorine, bromine, iodine.

Heteroaryl is a 5- or 6-membered aromatic ring containing one or two heteroatoms selected from O, N or S. In particular, it may be furan, pyrrole, thiophene, imidazole, pyrazole, pyridine, pyrimidine, pyrazine.

In one embodiment, the present invention is directed to a thia-aza-pryrimidine of formula (II):

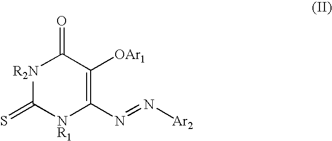

wherein R₁ and R₂ are independently selected from hydrogen or C₁₋₆alkyl that may be optionally substituted with halogen; Ar₁ is selected from the group consisting of optionally substituted tetrahydropyran, tetrahydrothiopyran, dithiane, aryl or heteroaryl; Ar₂ is an optionally substituted aryl group.

Preferably, R₁ and R₂ are the same or different and are hydrogen or C₁₋₆alkyl, optionally substituted by halogen; and Ar₁ and Ar₂ may be the same or different and are an aryl group optionally substituted with one or two C₁₋₆alkyl groups. More preferably, R₁ and R₂ are hydrogen and Ar₁ and Ar₂ are an aryl group independently optionally substituted with one or two C₁₋₆alkyl groups.

In a further embodiment, the present invention is directed to a compound of formula (III):

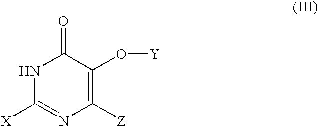

wherein X is hydrogen, C₁₋₄alkyl, S—CH₃, SH; Z is (a) NH₂; (b) hydrogen (c) —N═N—Ar, Ar being an optionally substituted aryl group; Y is hydrogen, optionally substituted tetrahydropyran or aryl. Preferably, Y is hydrogen, X is hydrogen, C₁₋₄alkyl, S—CH₃ or SH; and Z is NH₂.

The invention is further directed to a process for synthesizing a compound of formula (II), comprising:

(a) reacting formic ethyl formate with α-aryloxy-acetic acid ethylester in the presence of a base and thiourea or N,N′-substituted thiourea to yield an optionally substituted thia-pyrimidine of formula (II-a), according to the following reaction scheme:

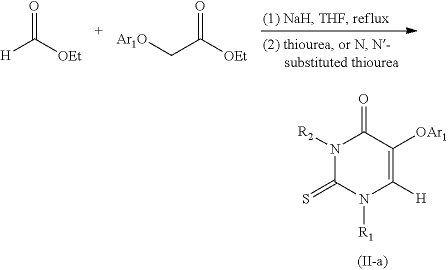

(b) reacting the optionally substituted thia-pyrimidine of formula (II-a) with aryldiazonium chloride, the aryl group being optionally substituted, to yield the thiaaza-pyrimidine of formula (II), according to the following reaction scheme:

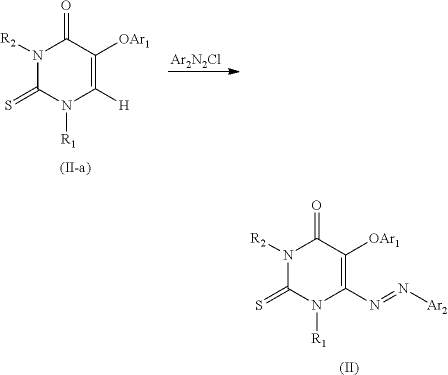

wherein R₁, R₂, Ar₁ and Ar₂ are as defined above. Substituted thiourea is a thiourea substituted by one or two C₁₋₆alkyl groups that may be optionally substituted with halogen.

The invention is further directed to a process for the manufacture of a pyrimidine of formula (III-a), comprising:

(a) reacting ethyl formate and the ethylester of the appropriate α-oxy acetic acid derivative in the presence of sodium hydride and C₁₋₄C(═O)NH₂ or thiourea to yield a compound of formula (IV), according to the following reaction scheme:

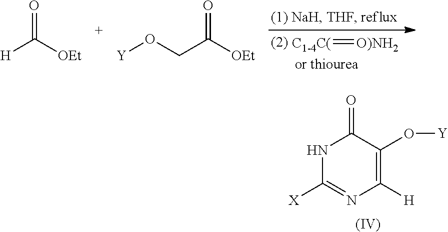

wherein X is CH₃ or SH and Y hydrogen, optionally substituted tetrahydropyran or aryl;

(b) hydrolyzing a compound of formula (IV) to yield a compound of formula (III-a), according to the following reaction scheme:

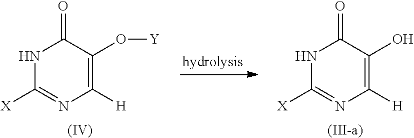

wherein X is C₁₋₄ or SH.

The invention is further directed to a process for the manufacture of a pyrimidine of formula (III-b), comprising:

reacting a compound of formula III-a as defined above, wherein X is SH; with Raney Ni to yield a compound of formula (III-b) according to the following reaction scheme:

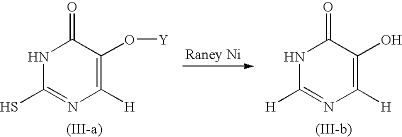

The invention is further directed to a process for the manufacture of a pyrimidine of formula (III-c), wherein X is SCH₃, Y and Z are hydrogen, comprising: reacting a compound of formula (III-a) of claim 7 wherein X is SH with (CH₃)₂SO₄ to yield a compound of formula (III-c) according to the following reaction scheme:

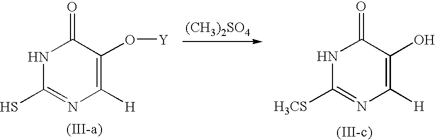

The invention is further directed to a process for the manufacture of a compound of formula (III-d) wherein X is hydrogen, SH, SCH₃ or C₁₄, Y is H and Z is —N═NAr₂ comprising:

reacting any one of compounds of formulae (III-a), (III-b), (III-c) with a diazotizing reagent of formula —N═NAr₂ Ar₂ as defined above, according to the following reaction scheme:

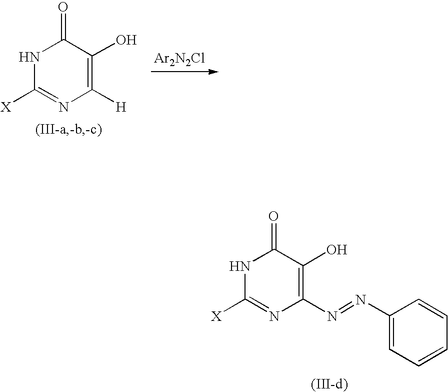

The invention is further directed to a process for the manufacture of a compound of formula III, wherein X is hydrogen, SH, SCH₃ or C₁₋₄, Y is hydrogen and Z is NH₂, comprising reacting a compound of formula III-d with dithionate according to the following reaction scheme:

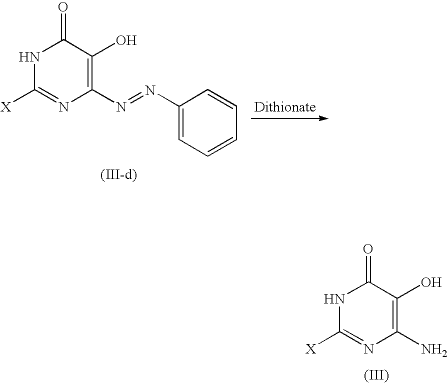

Compounds of formulae III-a. II-b, III-c or III-d or the compound of formula III wherein X is hydrogen, SH, SCH₃ or C₁₋₄, Y is hydrogen and Z is NH₂ being novel are also being part of the present invention.

The invention further relates to topical formulations providing protection for skin from the hazardous effects of ultra violet irradiation, in particular, UVA and UVB irradiation, comprising an effective amount of a compound of formulae I-III together with suitable adjuvants. Such topical formulations may further comprise at least one additional sun-protecting agent. The additional sun-protecting agent may be selected from the group consisting of organic or inorganic sun protecting agent. Non limiting examples of the at least one additional sun protecting agent are derivatives of anthranilates, benzophenones, camphors, cinnamates, dibenzoylmethanes, p-aminobenzoates, salicylates, zinc oxide, titanium dioxide and mixtures thereof.

The invention still further relates to the use of an effective amount of a compound of formulae I-III, optionally together with at least one additional sun-protecting agent for the preparation of a sunscreen formulation providing protection from ultra violet irradiation, in particular, UVA and UVB irradiation.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 depicts two prior art molecules used in comparison experiments reported herein.

FIG. 2 depicts Hammett correlations: Initial rate of dissociation vs. σₚ (filled squares) and σₚ⁺ (empty squares) for the autoxidation of compounds 8a-e in air saturated solutions at pH 7.0 and 25° C. p value for σₚ is −2.40, r²=0.5868, ρ value for σₚ⁺ is −1.28, r²=0.949.

FIG. 3 depicts the Ultra violet spectrum of a compound of formula (II).

### DETAILED DESCRIPTION OF EMBODIMENTS

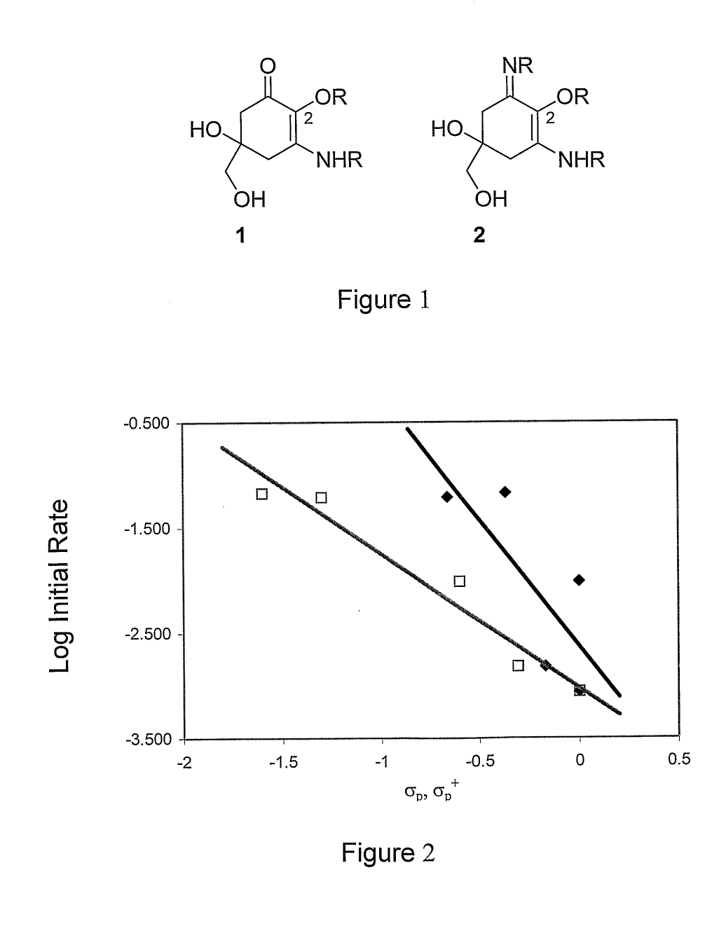

As mentioned above, the present invention is directed to new pyrimidine derivatives possessing unique structural features of enol-amines similar to those found in amino reductones and further having unique ultra violet absorption characteristics. Several amino reductones occur naturally and are responsible for life-threatening hemolytic episodes in favism. On the other hand, amino reductones may also be useful sunscreens (sun-protecting agents). The amino reductone structure i.e., enolamine, occurs in a number of natural products. For instance, several species of ancient organisms (fungi, cyanobacteria and lichens) produce UV absorbing metabolites such as MAA's (mycosporine like amino acids, FIG. 1) that are characterized by a cyclohexenone 1 or cyclohexenimine 2 chromophore conjugated with the nitrogen substituent of an amino acid or its imino alcohol and having absorption maxima ranging from 310 to 360 nm.

MAA's can be considered as potential sun-protecting agents as their conjugated amino enolic chromophore has both broad absorption in the UV region, and antioxidant properties desirable in a sun blocker. Certain pyrimidine derivatives, i.e., isouramil (6-amino-2,5-dihydroxypyrimidin-4-one; A_{d} in Scheme 1) and divicine (2,6-diamino-5-hydroxypyrimidin-4-one; Ae in scheme 1) incorporate the amino reductone group. They are found in beans as glycosides and are thought to be the causative agents in favism. A synergistic cytotoxicity has been demonstrated between carboplatin and divicine on murine erythroleukemic cells. Divicine also enhances in-vitro and in-vivo lipopolysaccharide-induced release of tumor necrosis factor (TNF).

In accordance with the present invention there exists a marked dependency of the autoxidation rate of five pyrimidine derivatives 8a-e (Scheme 1) on the electron releasing power of the substituent at C2 leading to a route for controlling the oxidation rate of such compounds in their use, in particular as sun screening agents. In order to examine their stability, kinetic studies of their auto-oxidation were carried, in particular in comparison to other known amine reductones.

Compounds 8a-c, of the present invention differ from isouramil (8d) and divicine (8e) only at C2. All five compounds were subjected to autoxidation under neutral conditions giving presumably H₂O₂ as shown in the following Scheme 1:

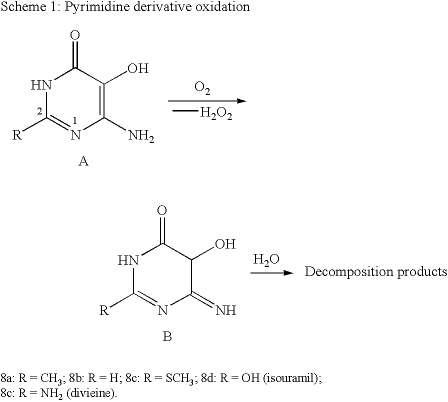

The autoxidation rate was measured in air-saturated buffer phosphate solutions 0.05 M at pH 7 and 25° C. The solutions contained 1 mM EDTA to minimize catalysis of the oxidation by trace metallic cations. The rate of autoxidation was measured spectrophotometrically by following the decrease of the UV absorbance of the pyrimidines at their respective λ max (Table 1).

TABLE 1
Spectral properties and initial autoxidation rates for
compounds Aa-e.

| Substituent R | λₘₐₓ nm (ε) | Initial rate μM/sec | σₚ^{a} | σₚ^{+a} |
|---|---|---|---|---|
| H | 274 (13000)^{b} | 0.00087^{c} | 0 | 0 |
| CH₃ | 275 (16200)^{b} | 0.0015^{c} | −0.17 | −0.31 |
| SCH₃ | 286 (10800)^{b} | 0.0097^{c} | 0 | −0.6 |
| OH | 280 (14100)^{d} | 0.067^{e} | −0.37 | −1.6^{f} |
| NH₂ | 285 (9800)^{d} | 0.061^{e} | −0.66 | −1.3 |

^{a}Cookell, S. C. (1998). Ultraviolet radiation, evolution and the π-electron system. Biological Journal of the Linnean Society, 63, 449-457.
^{b}This work.
^{c}This work; air saturated 0.05M phosphate buffer pH = 7.0, 1 mM EDTA, 25° C., [Pyrimidine] = 2.4 × 10⁻⁵ M. reference.
^{e}Calculated from (Sinha, R. P., Klisch, M., Grongier, M & Hader, D. P. (1998). Ultra violet-absorbing/screening substances in cyanobacteria, phytoplankton and macroalgae. J. Photochem. Photobiol. B: Biol., 47, 83-94) see text.
^{f}σₚ⁺ for OH from (R. P. Sinha, N. K. Ambasht, J. P. Sinha, M. Klisch and D. P. Häder. UV-B-induced synthesis of mycosporine-like amino acids in three strains of Nodularia (cyanobacteria), J. Photochem. Photobiol. B: Biol., 2003, 71, 51-5).

All three compounds 8a-c of the present invention showed much slower oxidation rates compared with isouramil and divicine. This is probably the reason why there was no transient appearance of an absorption maximum around 240-255 nm which is assumed to be due to the intermediacy of oxidized pyrimidine B (Scheme 1), and was found in the autoxidation of isouramil, divicine and related systems. The decrease in the absorbance of 8a-c, followed a reasonably pseudo first order reaction in the pyrimidine concentration (the dissolved oxygen concentration was at least 13 times higher). However, it was found (Winterbourn, C. C.; Cowden, W. B.; Sutton, H. Biochem. Pharmacol. 1989, 38, 611-618) that the reaction mechanism is complex and involves radical intermediates and chain reactions and that the dependency on the pyrimidine concentration is far from simple. Therefore, in accordance with the present invention a different approach was adapted that uses the measured initial reaction rates for checking the quantitative dependency of the oxidation rate on the electron releasing power of the substituent in the C2 position. The results for compounds 8a-c of the present invention are summarized in Table 1. Table 1 also contains the initial rates for the reactions of isouramil and divicine (8d and 8e) calculated from Winterbourn, C. C.; Cowden, W. B.; Sutton, H. Biochem. Pharmacol. 1989, 38, 611-618; (aerated 0.05M phosphate buffer, pH 7, 23° C., 50 nM DTPA and similar to our pyrimidine concentrations) using ΔH of 60.2 kJ/mole (Chevion et al.^{5a}). A plot of the logarithmic values of the initial rates against Hammett σₚ constants did not give a reasonable correlation. However, the correlation was greatly improved upon use of σₚ⁺ values, ρ value is −1.28 (r²=0.949) (FIG. 2).

The following complex chain mechanism (Scheme 2) was suggested (Winterbourn, C. C.; Cowden, W. B.; Sutton, H. Biochem. Pharmacol. 1989, 38, 611-618; Winterbourn, C. C.; Munday, R. Free Rad. Res. Commun. 1990, 8, 287-293) to account for the autoxidation rates of isouramil and divicine. DH₂ stands for the reduced pyrimidine, DH for the pyrimidine radical (probably structure 10) and D for the oxidized pyrimidine, most probably having structure B (Scheme 1). It is reasonable to expect the same mechanism for the compounds 8a, 8b and 8c (Scheme 1) of the present invention.

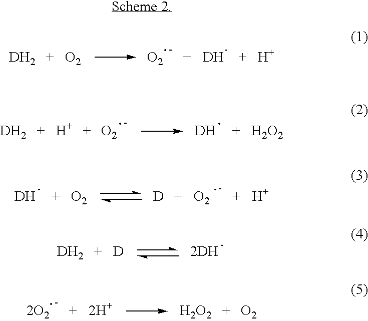

The observed initial reaction rates must be the result of a complex combination of the rates of the individual steps detailed in scheme 2. The observed initial rate surely reflects the rate of the first initiation step (1) as well as the rates of the rate determining propagation steps (2) and (4). Stabilizing the generated radical DH. will enhance the rate of the above three steps. The formation of DH. from DH₂ lowers the electron density on the oxygen atom and that explains the enhanced autoxidation rate with the electron releasing power of the substituent at position 2. The good correlation with ρₚ⁺ as reported herein clearly indicates the role of resonance and partial distribution of charge in stabilizing the generated pyrimidine radical.

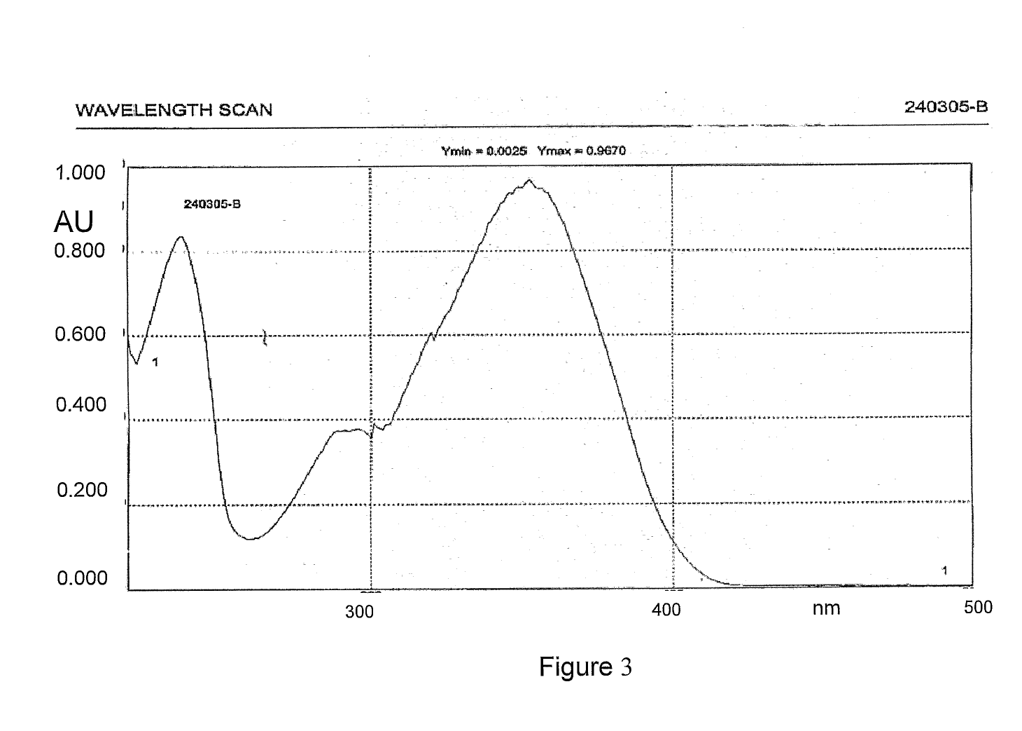

The compounds of the present invention possess absorption characteristics in the UV region. In particular, the compound of formula (II) exhibited an extraordinary and broad absorption covering wavelengths between 270-410 nm with a λₘₐₓ of 353 nm (FIG. 3). Absorption properties of compounds of formula (III) are displayed in Table 1.

Consequently, the compounds (I-III) of the present invention may be used as sun protecting agents in an appropriate topical formulation comprising suitable additives known for sun protection lotions. None limiting additives are selected from oils, aqueous additives, surfactants, emulsifiers.

Alternatively, the topical sun protecting formulation of the present invention may further comprise at least one additional organic or inorganic sun protecting agent. Non limiting examples of the at least one additional sun protecting agent are derivatives of anthranilates, benzophenones, camphors, cinnamates, dibenzoylmethanes, p-aminobenzoates, salicylates, zinc oxide, titanium dioxide and mixtures thereof.

The sun screen formulations of the present invention may also be encapsulated in appropriate encapsulating agent thus rendering their environment hydrophobic and aiding in dispersion on the skin.

### EXPERIMENTAL

The numbering of the compounds whose synthesis is given in examples 1-1H are depicted from the following shortened Scheme 3:

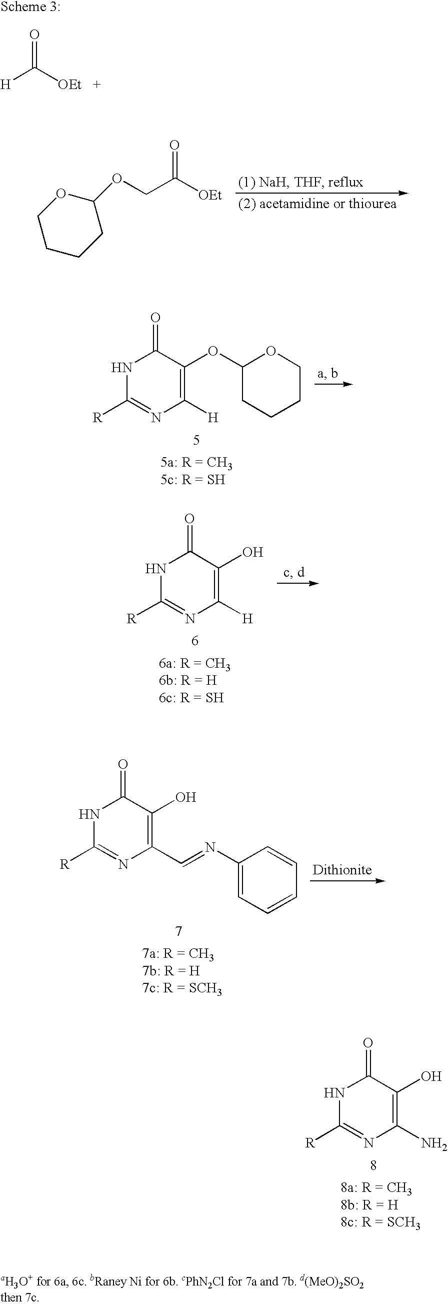

### Example 1

### 2-Methyl-5-(tetrahydro-2H-pyran-2-yloxy)pyrimidin-4(3H)-one 5a

To a suspension of sodium hydride (4.60 g in 55-60% paraffin oil), dry ether (50 cm³) and dry ethyl formate (7.84 g) were added. Then (tetrahydropyran-2-yloxy)-acetic acid ethyl ester (20 g) was added dropwise under continuous stirring. After the mixture was refluxed for 2 h, acetamidine (4.3 g) was added. After the removal of ether from the reaction mixture, the remained ethanolic solution was refluxed for 4 h. Then the mixture was cooled and the volatile solvents were removed by rotory-evaporator. The residue was redissolved in water and filtered. The filtrate was acidified by acetic acid in an ice bath and the white precipitate was filtered, washed with water and dried under reduced pressure at 100° C. (9.0 g, 58%); (Found: C, 55.25; H, 47.54; N, 13.19. Calc. for C₁₀H₁₆N₂O₃: C, 55.59; H, 7.60; N, 13.20%). Mp 149-151° C.; δ_{H} (300 MHz; DMSO_{d}) 1.23-2.03 (6H, m), 2.27 (3H, s), 3.23-4.04 (2H, m), 5.47 (1H, bs), 7.60 (1H, s), 10.73 (1H, bs). vₘₐₓcm⁻¹ 1670, 1610, 1385, 1310, 1205, 1190, 1120, 980, 910, 820, 775 and, 740; MS (EI): m/z (%) 210 (100, M⁺, C₁₀H₁₄N₂O_{3,}), 126 (M⁺, —C₅H₈O), 125 (M⁺-C₅H₉).

### Example 2

### 5-Hydroxy-2-methylpyrimidin-4(3H)-one 6a

A few crystals of p-toluenesulfonic acid were added to a solution of 1a (6.57 g) in hot methanol. After cooling the mixture in an ice bath, white crystals were formed. The crystals were filtered and dried under vacuum at 100° C. (2.5 g, 63%); Mp>300° C.; δ_{H} (300 MHz; DMSO_{d}) 2.2 (3H, s), 7.27 (1H, s), 9.27 (1H, bs), vₘₐₓcm⁻¹ 3300, 1670, 1620, 1420, 1380, 1245, 1110, 1020, 875 and 780; MS (EI): m/z (%) 126 (100, C₅H₆N₂O₂, M⁺), 108 (M⁺, —H₂O), 100 (M⁺, −26).

### Example 3

### 5-Hydroxy-2-methyl-6-phenylazo-3H-pyrimidin-4-one 7a

Diazotation of aniline (1.17 g) was done in hydrochloric acid (3.9 cm³) and water (8 cm³) by addition of sodium nitrite (0.87 g) in water (6 cm³) at 0-5° C. Then sodium acetate (3.1 g) was added slowly under continuous stirring, followed by the addition of a solution of 6a (1.59 g) in 10% sodium hydroxide (10.4 cm³). After stirring for 30 min, the reaction mixture was left for overnight at 4° C. Then the reaction was warmed to 40° C. for 1 h and filtered. The red crystals formed were washed and dried under vacuum at 100° C. (1.61 g, 56%); (Found: C, 57.60; H, 4.22. Calc. for C₁₁H₁₀N₄O₂: C, 57.89; H, 4.35%); Mp 243-245° C.; δ_{H} (300 MHz; DMSO_{d}) 1.95 (3H, s), 6.44-7.73 (5H, m), 11.19 (1H, bs), 11.64 (1H, bs); vₘₐₓcm⁻¹ 3220, 3180, 1710, 1670, 1600, 1520, 1470, 1430, 1360, 1280, 1250, 1050, 800, 775, 715, 700 and 660; MS (EI): m/z (%) 230 (25, C₁₁H₁₀N₄O₂, M⁺), 105 (100, C₆H₅N₂⁺).

### Example 4

### 6-Amino-5-hydroxy-2-methylpyrimidin-4(3H)-one 8a

A solution of 7a (1.61 g) in water (15 cm³) was heated to 60-70° C., an excess of sodium dithiollite was added to the solution in batches until a bright yellow color was obtained and the solution was cooled in an ice bath. The white crystals formed were washed with water and dried under vacuum at 100° C. (0.44 g, 44%); (Found: C, 42.53; H, 4.98; N 29.46. Calc. for C₅H₇N₃O₂: C, 42.55; H, 4.96; N 29.79%); Mp>300° C.; δ_{H} (300 MHz; DMSO_{d}) 2.13 (3H, s), 5.36 (2H, bs), 7.85 (1H, bs), 11.66 (1H, bs); vₘₐₓcm⁻¹ 3420, 3320, 3160, 1600, 1440, 1380, 1280, 1210, 1020, 990, 900, 790 and 770; MS (EI): m/z (%) 141 (100, C₅H₇N₃O₂, M⁺).

### Example 5

### 2-Mercapto-5-(tetrahydro-pyran-2-yloxy)-3H-pyrimidin-4-one 5c

Identical procedure to the synthesis of 5a, except for the addition of thiourea (15.29 g) instead of acetamidine. White crystals were obtained. (25.0 g, 54.5%); (Found: C, 47.15; H 5.38; S 14.49. Calc. for C₉H₁₂N₂O₃S: C, 47.37; H, 5.26; S 14.00%); Mp>300° C.; δ_{H} (300 MHz; DMSO_{d}) 0.8-1.97 (6H, m), 3.23-3.80 (2H, m), 5.27 (1H, bs), 7.16 (1H, d, J_{HNCH}=6.0 Hz), 10.8 (1H, bs), 11.3 (1H, bs); vₘₐₓcm⁻¹ 3150, 3080, 1630, 1570, 1250, 1200, 1180, 1150, 1110, 1020, 980, 940, 900, 870, 810 and 670; MS (EI): m/z (%) 147 (100, M⁺−81).

### Example 6

### 5-Hydroxy-2-mercapto-3H-pyrimidin-4-one 6c

A suspension of 5c (6.0 g) in 1M H₂SO₄ (30 cm³) was stirred for 2 h. Then the product was filtered and washed with water, methanol and ether and dried under vacuum at 100° C. (3.2 g, 84.4%); (Found: C, 33.53; H, 2.60; N 19.30; S 22.96. Calc. for C₄H₄N₂O₂S: C, 33.33; H, 2.78; N 19.44; S 22.22%); Mp>300° C.; δ_{H} (300 MHz; DMSO_{d}) 6.97 (1H, d, J_{HNCH}=6.0 Hz), 9.60 (1H, bs), 10.27 (1H, bs); vₘₐₓcm⁻¹ 3240, 3100, 1660, 1580, 1400, 1290, 1230, 1170, 1140, 890, 820, 760, 750 and 690; MS(EI): m/z (%) 144 (100, C₄H₄N₂O₂S, M⁺).

### Example 7

### 5-Hydroxy-2-methylsulfanyl-6-phenylazo-3H-pyrimidin-4-one 7c

6c (2.9 g) was dissolved in a solution of sodium hydroxide (1.8 g) in water (12 cm³) and heated to 40° C. Dimethylsulfate (3.0 g) was added dropwise while vigorously stirring and then the mixture was cooled in an ice bath and filtered. The filtrate was acidified with concentrated hydrochloric acid and was left overnight at 4° C. The formed crystals were filtered, successively washed with water, methanol and ether and dried under vacuum at 100° C. The 2-methylthio-4,5-dihydroxypyrimidine formed (1.4 g) was subjected to the same diazotization procedure as 3a. (1.5 g, 74.0%); Mp 213-215° C.; δ_{H} (300 MHz; DMSO_{d}) 2.73 (3H, s); 6.97-7.83 (5H, m); 10.73 (1H, bs). vₘₐₓcm⁻¹ 3480, 3200, 3100, 1710, 1660, 1590, 1510, 1450, 1250, 1150, 1030, 990, 770, 750, 690 and 640; MS (EI): m/z (%) 262 (10, C₁₁H₁₀N₄O₂S, M⁺), 105 (100, C₆H₅N₂⁺), 91 (C₆H₅N⁺), 77 (C₆H₅⁺).

### Example 8

### 6-Amino-5-hydroxy-2-(methylthio)pyrimidin-4-(3H)-one 8c

Identical to the procedure for the synthesis of 8a, except for the use of 7c (1.0 g) instead of 7a. White crystals were obtained. (0.43 g, 66%); (Found: C, 34.27; H, 4.08; N 24.21; S 19.00. Calc. for C₅H₇N₃O₂S: C, 34.38; H, 4.05; N 24.28; S 18.50%); Mp 243-245° C.; δ_{H} (300 MHz; DMSO_{d}) 2.45 (3H, s); 5.87 (2H, bs), 7.90 (1H, bs); vₘₐₓcm⁻¹ 3250, 3380, 1640, 1600, 1570, 1410, 1330, 1240, 970, 830 and 760; MS (EI): m/z (%) 173 (100, CsH₇N₃O₂S, M⁺).

### Example 9

### 5-Hydroxypyrimidin-4(3H)-one 6b

To a solution of water (76 cm³) and concentrated aqueous ammonia (7.6 cm³), 5c (11.0 g) was added followed by the addition of Raney nickel (40.0 g). The mixture was refluxed for 4 h then it was cooled and filtered. All the volatile solvents were removed by rotor-evaporator and the residue was re-dissolved in methanol. After addition of ether, pink crystals precipitated out of the solution, and were dried under vacuum at 100° C. (2.5 g, 46%); Mp 265-267° C.; δ_{H} (300 MHz; DMSO_{d}) 7.40 (1H, s), 7.67 (1H, s); vₘₐₓcm⁻¹ 1640, 1600, 1360, 1300, 1270, 1100, 930, 880, 790, 780 and 615; MS (EI): m/z (%) 112 (100, C₄H₄N₂O₂, M⁺).

### Example 10

### 5-Hydroxy-6-phenylazo-3H-pyrimidine-4-one 7b

Identical to the procedure for the synthesis of 7a except for the addition of 6b (1.12 g) instead of 6a. White crystals were obtained. (1.9 g, 88%); Mp 244-245° C.; δ_{H} (300 MHz; DMSO_{d}) 6.83-7.76 (6H, m), 11.88 (1H, bs); vₘₐₓcm⁻¹ 3490, 3290, 1700, 1650, 1615, 1600, 1590, 1500, 1450, 1300, 1240, 1170, 1120, 1015, 900, 875, 750, 730, 680, 660, 640, 660 and 640; MS (EI): m/z (%) 216 (15, C₁₀H₈N₄O₂, M⁺), 105 (100, C₆H₅N₂⁺), 91 (C₆H₅N⁺), 77 (C₆H₅⁺).

### Example 11

### 6-Amino-5-hydroxypyrimidin-4(3H)-one 8b

Identical to the procedure for the synthesis of 8a except for the addition of 7b (1.0 g) instead of 7a. (0.45 g, 76%); (Found: C, 38.02; H, 3.75; N, 33.35. Calc. for C₄H₅N₃O₂: C, 37.80; H, 3.94; N, 33.07%) Mp>300° C.; δ_{H} (300 MHz; DMSO_{d}) 5.83 (2H, bs), 7.56 (1H, s), 11.79 (1H, bs); vₘₐₓcm⁻¹ 3470, 3140, 1670, 1640, 1620, 1440, 1370, 1250, 1170, 1010, 890, 810, 770 and 650; MS (EI): m/z (%) 127 (100, C₄H₅N₃O₂, M⁺).

1. A pyrimidine derivative of formula (I): 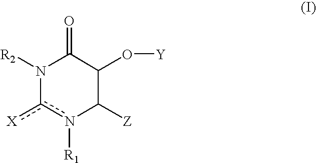
wherein [symbol]
denotes an optional double bond, where one of the two optional double bonds being a double bond and the other a single bond; R₁ is null, hydrogen or C₁₋₆alkyl which may be optionally substituted with halogen; R₂ is hydrogen or C₁₋₆alkyl which may be optionally substituted with halogen; X is hydrogen, C₁₋₆alkyl, S—CH₃, SH or ═S; Y is hydrogen, optionally substituted tetrahydropyran, tetrahydrothiopyran, dithiane, or an optionally substituted aryl or heteroaryl; and Z is (a) NH₂; (b) hydrogen (c) —N═NAr, Ar being optionally substituted aryl group.

2. A thia-aza-pryrimidine according to claim 1 of formula (II): 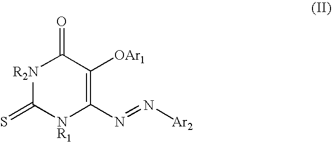
wherein R₁ and R₂ are independently selected from hydrogen or C₁₋₆alkyl that may be optionally substituted with halogen; Ar₁ is selected from the group consisting of optionally substituted tetraliydropyran, tetrahydrothiopyran, ditliiane, aryl or heteroaryl; and Ar₂ is an optionally substituted aryl group.

3. A thia-aza-pryrimidine according to claim 2, wherein R₁ and R₂ are the same or different and are hydrogen or C₁₋₆alkyl; and Ar₁ and Ar₂ may be the same or different and are an aryl group optionally substituted with one or two C₁₋₆alkyl groups.

4. A pyrimidine according to claim 1 of formula (III): 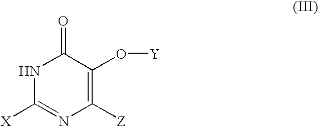
wherein X is hydrogen, C₁₋₄alkyl, S—CH₃, SH; Z is (a) NH₂; (b) hydrogen (c) —N═NAr, Ar being an optionally substituted aryl group; Y is hydrogen, optionally substituted tetrahydropyran or aryl.

5. A pyrimidine according to claim 4 wherein X is hydrogen, methyl, ethyl or propyl, SH or S—CH₃; Y is hydrogen; and Z is NH₂.

6. A process for the manufacture of a thia-aza-pyrimidine of formula (II) as defined in claim 2, comprising:
(a) reacting formic ethyl formate with [alpha]-aryloxy-acetic acid ethylester in the presence of a base and thiourea or N,N′-substituted thiourea to yield an optionally substituted thia-pyrimidine of formula (II-a), according to the following reaction scheme: 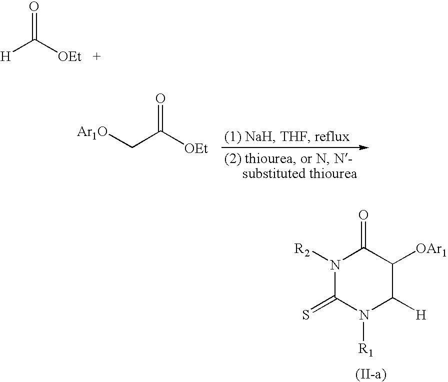
(b) reacting the optionally substituted thia-pyrimidine of formula (II-a) with aryldiazonium chloride, the aryl group being optionally substituted, to yield the thiaaza-pyrimidine of formula (II), according to the following reaction scheme:

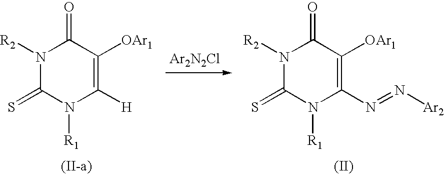

7. A process for the manufacture of a pyrimidine of formula (III-a), comprising:
(a) reacting ethyl formate and the ethylester of the appropriate a-oxy acetic acid derivative in the presence of sodium hydride and C₁₋₄C(═O)NH₂ or thiourea to yield a compound of formula (IV), according to the following reaction scheme: 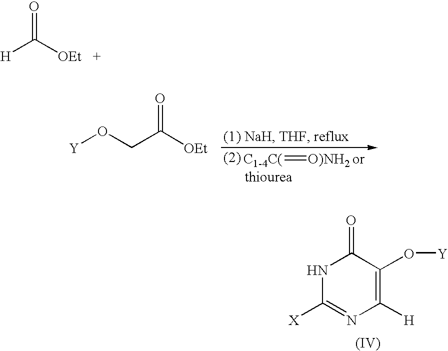
wherein X is CH3 or SH and Y hydrogen, tetrahydropyran or aryl;
(b) hydrolyzing a compound of formula (IV) to yield a compound of formula (III-a), according to the following reaction scheme: 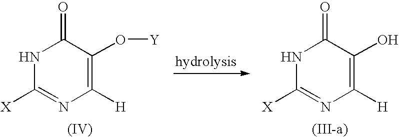
wherein X is C₁₋₄ or SH.

8. A process for the manufacture of a pyrimidine of formula (III-b), comprising: reacting a compound of formula III-a of claim 7, wherein X is SH with Raney Ni to yield a compound of formula (III-b) according to the following reaction scheme:

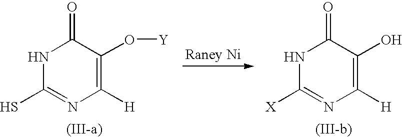

9. A process for the manufacture of a compound of formula (III-c) wherein X is SCH₃, Y and Z are hydrogen, comprising:
reacting a compound of formula (III-a) of claim 7 wherein X is SH with (CH₃)₂SO₄ to yield a compound of formula (III-c) according to the following reaction scheme:

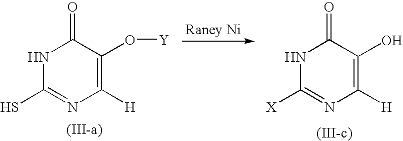

10. A process for the manufacture of a compound of formula (III-d) wherein X is hydrogen, SH, SCH₃ or C₁₋₄, Y is H and Z is —N═NAr₂ comprising:
reacting any one of compounds of formulae (III-a), (III-b), (III-c) as defined in claim 7 with a diazotizing reagent of formula —N═NAr₂, Ar₂ being an optionally substituted aryl group: Y is hydrogen, optionally substituted tetrahydropyran or aryl; according to the following reaction scheme:

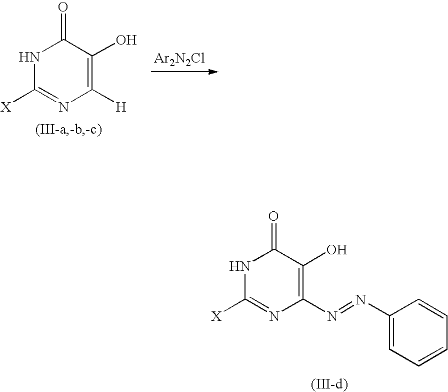

11. A process for the manufacture of a compound of formula III, wherein X is hydrogen, SH, SCH₃ or C₁₋₄, Y is hydrogen and Z is NH₂, comprising reacting a compound of formula III-d with dithionate according to the following reaction scheme:

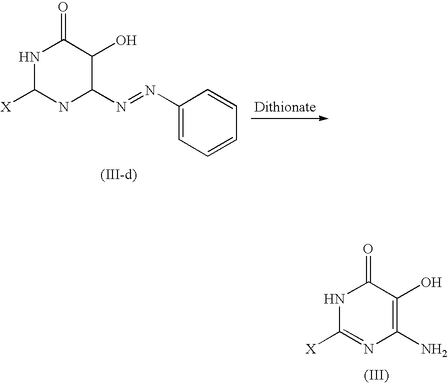

12. A compound of formulae III-a, III-b, III-c or III-d.

13. A compound of formula III wherein X is hydrogen, SH, SCH₃ or C₁₋₄, Y is hydrogen and Z is NH₂.

14. A topical formulation for providing protection from ultra violet irradiation comprising an effective amount of a compound of formula (I) of claim 1 together with suitable additive or excipient.

15. A topical formulation for providing protection from ultra violet irradiation comprising an effective amount of a compound of formula (II) of claim 2 together with suitable additive or excipient.

16. A topical formulation for providing protection from ultra violet irradiation comprising an effective amount of a compound of formula (III) of claim 4 together with suitable additive or excipient.

17. A topical formulation according to claim 14 further comprising an additional sun-protecting agent.

18. A topical formulation according to claim 17, wherein said additional sun-protecting agent is chosen from the group comprising of derivatives of anthranilates, benzophenones, camphors, cinnamates, dibenzoylmethanes, p-aminobenzoates, salicylates, zinc oxide, titanium dioxide or mixtures thereof.
